SEQUENCE LISTING (26 sequences)

SEQ ID NO: 1 - protein, 7 aa, Artificial Sequence
  Transportation peptide
PKKKRKV

SEQ ID NO: 2 - protein, 4 aa, Artificial Sequence
  Transportation peptide
RRRR

SEQ ID NO: 3 - protein, 11 aa, Artificial Sequence
  Transportation peptide
PKKKRKVRRRR

SEQ ID NO: 4 - protein, 5 aa, Artificial Sequence
  Specificity mediating peptide
PLGLA

SEQ ID NO: 5 - protein, 5 aa, Artificial Sequence
  Specificity mediating peptide
PLGVA

SEQ ID NO: 6 - protein, 10 aa, Artificial Sequence
  Protease recognition sequence
LVLASSSVGY

SEQ ID NO: 7 - protein, 11 aa, Artificial Sequence
  Protease recognition sequence
LVLASSSFGYS

SEQ ID NO: 8 - protein, 10 aa, Artificial Sequence
  Protease recognition sequence
GVSQNYPIVG

SEQ ID NO: 9 - protein, 10 aa, Artificial Sequence
  Protease recognition sequence
GVVQASCRLA

SEQ ID NO: 10 - protein, 8 aa, Artificial Sequence
  Transportation peptide
PPKKKRKV

SEQ ID NO: 11 - protein, 6 aa, Artificial Sequence
  Transportation peptide
KRRRER

SEQ ID NO: 12 - protein, 10 aa, Artificial Sequence
  Transportation peptide
VQRKRQKLMP

SEQ ID NO: 13 - protein, 10 aa, Artificial Sequence
  Transportation peptide
VQRKRQKLMP

SEQ ID NO: 14 - protein, 8 aa, Artificial Sequence
  Transportation peptide
SKKKKTKV

SEQ ID NO: 15 - protein, 8 aa, Artificial Sequence
  Transportation peptide
GRKRKKRT

SEQ ID NO: 16 - protein, 11 aa, Artificial Sequence
  Transportation peptide
GKKKKRKREKL

SEQ ID NO: 17 - protein, 8 aa, Artificial Sequence
  Transportation peptide
ERKKRRRE

SEQ ID NO: 18 - protein, 7 aa, Artificial Sequence
  Transportation peptide
FKKFRKF

SEQ ID NO: 19 - protein, 7 aa, Artificial Sequence
  Transportation peptide
KPPSKKR

SEQ ID NO: 20 - protein, 11 aa, Artificial Sequence
  Transportation peptide
RPRYAKRRIRL

SEQ ID NO: 21 - protein, 7 aa, Artificial Sequence
  Transportation peptide
RRRKRRR

SEQ ID NO: 22 - protein, 16 aa, Artificial Sequence
  Transportation peptide
RQIKIWFQNRRMKWKK

SEQ ID NO: 23 - protein, 10 aa, Artificial Sequence
  Transportation peptide
RXRXRXRXRX

SEQ ID NO: 24 - protein, 11 aa, Artificial Sequence
  Transportation peptide
YGRKKRQRRRP

SEQ ID NO: 25 - protein, 7 aa, Artificial Sequence
  Transportation peptide
RHRHHRR

SEQ ID NO: 26 - protein, 6 aa, Artificial Sequence
  Transportation peptide
RKHRKH

(19) United States
(12) Patent Application Publication
(10) Pub. No.: US 2010/0267928 A1
(43) Pub. Date: Oct. 21, 2010

(54) ACTIVATABLE DIAGNOSTIC AND THERAPEUTIC COMPOUND

(76) Inventor: Stefan HECKL, Tuebingen (DE)

Correspondence Address: HARNESS, DICKEY & PIERCE, P.L.C., P.O. BOX 828, BLOOMFIELD HILLS, MI, 48303, US

(21) Appl. No.: 12/634,972

(22) Filed: Dec. 10, 2009

Related U.S. Application Data
(63) Continuation of application No. PCT/EP2008/004481, filed on Jun. 5, 2008.

(30) Foreign Application Priority Data
Jun. 12, 2007 (DE) 10 2007 028 090.6

(51) Int. Cl. C07K 14/00; C07K 7/08; C07K 7/06

(52) U.S. Cl. 530/325; 530/326; 530/327

(57) ABSTRACT

The present invention relates to a compound which can be used as a contrast medium and as a therapeutic agent, the use of the compound for manufacturing a diagnostic or therapeutic composition, a diagnostic and therapeutic composition which comprises the compound, and a method for the diagnostic and therapeutic treatment of a living being.

### CROSS-REFERENCE TO RELATED APPLICATIONS

This application is a continuation of copending international patent application PCT/EP 2008/004481 filed on Jun. 5, 2008 and designating the U.S., which was not published under PCT Article 21(2) in English, and claims priority of German patent application DE 10 2007 028 090 filed on Jun. 12, 2007, which is incorporated herein by reference.

### BACKGROUND OF THE INVENTION

1. Field of the Invention

The present invention relates to a compound which can be used as a contrast medium and as a therapeutic agent, the use of said compound for manufacturing a diagnostic or therapeutic composition, a diagnostic and therapeutic composition which comprises said compound, and a method for the diagnostic and therapeutic treatment of a living being.

2. Related Prior Art

Compounds and methods of these kinds are generally known in the art.

An objective of modern medicine and pharmacy is to develop improved therapeutic and diagnostic compounds or compositions, which can be used in a targeted manner, i.e. in such a manner, that after the administration, they find their areas or target structures in the organism, where they should develop their effect or should accumulate, autonomously.

Contrast media, such as gadolinium complexes, which are used as a matter of routine in clinical magnetic resonance imaging (MRI), such as Magnevist (Schering) or DOTAREM (Guerbet), after been administered into a patient, accumulate in the extracellular space and are not able to penetrate the cytoplasm of the cells; Prantner et al. (2003), Synthesis and characterization of a Gd-DOTA-D-permeation peptide for magnetic resonance relaxation enhancement of intracellular targets, Mol. Imaging 2(4): 333-41. When imaging e.g. tumors of the brain this results in considerable disadvantages. The tumor tissue is in many cases not precisely distinguishable from healthy or inflammatory tissue what results in a blurred imaging of the boundaries of the tumor. If such an extracellular contrast medium is administered during or directly after surgery the contrast medium flows along the intracellular space, opened by the surgeon, beyond the boundaries of the tumor, what strongly limits this technology; Okudera et al. (1994); Intraoperative CT scan findings during resection of glial tumours, Neurol. Res. 16(4): 265-7.

However contrast media which accumulate in the cytoplasm or even in the cell nucleus of tumor cells would enable a better imaging of the boundaries of the tumor in the magnetic resonance imaging.

The WO 01/08712 A2 describes a contrast medium containing gadolinium for a use in the magnetic resonance imaging, which has an extremely complex structure. The known contrast medium is provided to bind via so-called “target binding moieties” (TBM) to large proteins in the blood, such as albumin or fibrin, and to remain in the blood stream as long as possible. The known contrast medium neither can penetrate the cytoplasma nor the cell nucleus of biological cells.

Caravan et al. (2003), Gadolinium-binding helix-turn-helix peptides: DNA-dependent MRI contrast agents. Chem. Commun., 2574-2575, describe a peptidic DNA-binding contrast medium which consists of 33 amino acids in total, a centrally located segment which is derived from a calcium-binding EF-hand and is in the position to chelate gadolinium, and an N-terminal and C-terminal peptidic segment via which a binding of the construct to DNA can take place. The known contrast medium is in the position to enter the cytoplasm of cells, however not the cell nucleus. Further, the affinity of the known peptide to gadolinium is too low so that a biological application as a contrast medium is not possible due to the risk of the release of the toxic gadolinium ion into the organism.

The document WO 2004/050698 A2 discloses gadolinium containing contrast media for a use in the magnetic resonance imaging. These contrast media comprise apart from gadolinium 4 molecules of a chelating agent [(DTPH)₄] and a peptide which contains so-called NLS and TPU segments. The TPU segment (“transport peptide unit” or “transmembrane module”, respectively) is derived from penetratin, transportan or the HOX-B1 protein and mediates the permeability of the contrast medium through the cell membrane. The NLS segment (“nuclear localization sequence”) mediates the permeability of the known contrast medium through the membrane of a cell nucleus. According to the authors this contrast medium is, for these reasons, in the position to enter the cytoplasm and also the cell nucleus. The known contrast medium has the particular disadvantage that it is very large and complex in its construction. The disclosed compounds comprise molecular weights of about 4,700 kDa to 6,500 kDa. Due to the unfavorable weight ratio of the large TPU segment to the gadolinium containing signaling segment the emitting signal is negatively influenced in the magnetic resonance tomography. I.e. the emitted signal is relatively weak. The known contrast medium further comprises a bisulfite bridge. This has the disadvantage that the bisulfite bridge is already cleaved in the blood stream by bisulfite reductases which could result in an inactivation of the known contrast medium.

In the WO 2006/056227 A1a gadolinium-containing contrast medium for a use in the magnetic resonance tomography is proposed. The known peptidic contrast medium comprises, in its central position, a gadolinium which is chelated by DTPA, flanked toward the N- and C-terminals by short peptides which comprise a positive net charge. According to the authors by the flanking peptides a penetration of the cytoplasm and the cell nucleus or a penetration of the cell membrane and the cell nucleus membrane of biological cells is enabled. According to a modified configuration of the known contrast medium, which is designated as conjugate 8 or C8, respectively, to the C-terminus of the known compound, via a short peptide which comprises a cleavage side for the tumor-cell specific enzyme matrix metalloproteinase 2 (MMP-2), a negatively charged peptide is linked which neutralizes the charge and, in the consequence, also the function of the two positively charged peptides which flank the Gd-DTPA complex. The known conjugate modified in this manner is in the consequence, according to the authors, not in the position to penetrate non-transformed, i.e. healthy cells. Tumor cells secrete MMP-2 into their environment. If the known conjugate is present in the environment of such a tumor cell the cleavage site in the connecting peptide is recognized and cleaved. The negative charge of the linked peptide now can no longer neutralize the positive charge of the flanking peptides. The cleaved conjugate 8 should now be again in the position to penetrate the cells. Since the enzyme MMP-2 is only secreted by tumor cells the conjugate only enters tumor cells, however not healthy cells. The conjugate 8 known from the WO 2006/056227 A1 has, however, not been proven in practice. It has been shown that the neutralizing negatively charged peptide which, in essence, consists of glutamic acid residues, is neurotoxic to the body after its cleavage; cf. Garattini (2000), Glutamic Acid, Twenty Years Later, J. Nutr. 130(4S Suppl):901-9S. An application of the conjugate 8 in a human being is therefore excluded.

Peptides which are based on the same principle and can selectively penetrate cells or the cell nucleus of tumor cells, are described by Jiang et al. (2004), Tumor Imaging by Means of Proteolytic Activation of Cell-Penetrating Peptides, PNAS Vol. 101, No. 51, 17867-17872 (cf. also WO 2005/042034), and by Liu et al. (2007), Characteristics and In Vitro Imaging Study of Matrix Metalloproteinase-2 targeting Activable Cell-Penetrating Peptide, Natl. Med. J. China, Vol. 87, No. 4, 233-239. Also these peptides are selectively cleaved in the environment of tumor cells and release neurotoxic glutaminic acid containing peptides into the body. In addition, the peptide described by Jiang et al. only comprises a fluorescent marker, however not an X-ray-dense unit or such a unit which emits a signal in the magnetic resonance tomography, so that only superficial tumors can be visualized. Already for this reason an application in a human being is excluded.

Pharmaceutical substances, e.g. cytotoxic agents, which are currently used in the treatment of tumor diseases, are mostly unspecific and are directed against all rapidly dividing cells. This results in severe side effects, e.g. a damage of healthy tissues or cells, such as the hematopoietic system, the gonads or hair follicles, etc. The first step for the development of a cellularly targeted and tumor-specific agent would, therefore, be the provision of a compound which can penetrate the cytoplasm or the cell nucleus of a tumor cell, respectively, in a targeted manner and there develops its cytotoxic properties without damaging healthy cells.

### SUMMARY OF THE INVENTION

Against this background the problem underlying the invention is to pro-vide a diagnostically and/or therapeutically valuable compound by means of which the disadvantages of the compounds known in the prior art can be largely avoided. In particular such a compound should be provided which can be used as an improved contrast medium or a therapeutic active agent, and can penetrate the cytoplasm or the cell nucleus of tumor cells or virus-infected cells, respectively, in a targeted and selective manner, and there can develop signaling or cytotoxic properties. Such a compound should be producible, in a large scale, in a cost-effective manner.

This problem is solved by the provision of a compound which comprises:

a first unit (E1), comprising:

a first component providing a permeability through the cell membrane and the nuclear membrane (transportation component),

a metal complexing agent,

a second unit (E2), comprising:

a peptide comprising at least one cleavage side for tumor or virus specific proteases (specificity mediating peptide), and

a third unit (E3), comprising:
at least one metal complexing agent, wherein E1 is connected to E2 and E2 is connected to E3.

A compound designed according to this basic principle is fully solving the problem underlying the invention.

Transportation components which mediate a permeability through the cell membrane and the cell nucleus membrane are comprehensively described in the art. They encompass peptidic compounds, such as described in Martin and Rice (2007), Peptide-guided Gene Delivery, The AAPS Journal 9(1), E18-E29, and in Schwartz and Zhang (2000), Peptide-mediated cellular delivery, Current Opinion in Molecular Therapeutics 2(2); the content of these publications is incorporated herein by reference. Transportation components encompass also non-peptidic components, such as cortisone, progesterone or peroxisome proliferator-activated receptor (PPAR)-ligand. Non-peptidic components have the advantage that they are very small. The transportation components in general comprise a positive net charge, however can also be neutral.

Cell membrane and cell nucleus membrane permeability means that the compound according to the invention, under physiological conditions, can specifically penetrate the cytoplasm and the cell nucleus of intact cells. The permeability of the compound according to the invention through the cell membrane and the cell nucleus membrane can be measured in vitro in the cell culture in a relatively easy manner, e.g. by incubating a first aliquot of the compound in a tumor cell culture versus a second aliquot of a compound in a culture of healthy cells. The compound in the tumor cell culture can be found in the cell nucleus after a short time, whereas the compound in the culture of healthy cells cannot be found in the cell nucleus or only in a small amount due to an unspecific uptake.

Complexing agents for metals, such as gadolinium (Gd), gallium (Ga), manganese (Mn), iron (Fe), yttrium (Y) are comprehensively described in the prior art, and encompass e.g. tetraazacyclododecane tetraacetic acid (DOTA), diethylene triamine pentaacidic acid (DPTA), BOPTA, EOB-DTPA, DTPA-DMA, HP-DOBA, DTPA-BMEA, HIDA, DTDP, porphyrine, texaphyrine, etc. These complexing agents can be easily linked to peptides by routine methods, e.g. to lysine residues.

Cleavage sites for tumor or virus-specific proteases are comprehensively described in the prior art. It is known that several tumor or carcinoma cells express characteristic enzymes and secrete them into their cellular environment. For invading tumors they can digest the surrounding connective tissue to enable the penetration of the transformed cells of so far healthy tissue or organs. Glioblastomas express and secrete e.g. the matrix metalloprotease 2 (MMP-2). MMP-2 recognizes the amino acid sequence PLGVR or PLGLA. Mamma carcinomas express and secrete predominantly cathepsines. Cathepsine B recognizes the specific amino acid sequences HK and/or RR. Cathepsine D recognizes the sequence PIC(Et)FF, wherein “Et” refers to an ester branch. Cathepsine K recognizes and cleaves the specific amino acid sequence GGPRGLPG. Prostate carcinomas express and secrete predominantly the prostate-specific antigen (PSA). PSA recognizes and cleaves the amino acid sequence HS SKLQ. Other tumor cell specific enzymes and their specific recognition and cleavage sites are comprehensively described in the prior art. An overview is given in Hahn W. C. and Weinberg R. A. (2002), Rules for making human tumour cells, N. Engl. J. Med., 347: 1593-1603. The content of this publication is incorporated herein by reference.

It is also well known in the prior art that cells which were infected by viruses express and secrete virus-specific proteases. Cells infected by the herpes-simplex virus (HSV) secrete e.g. the HSV protease. The HSV protease recognizes and cleaves the amino-acid sequences LVLASSSVGY (SEQ ID No. 6) and (LVLASSSFGYS (SEQ ID No. 7). Cells which were infected by the human immuno-deficiency virus (HIV) express and secrete the HIV protease. The HIV protease recognizes and cleaves the amino acid sequence GVSQNYPIVG (SEQ ID No. 8). Cells which were infected by the cytomegalovirus (CMV), express and secrete a CMV protease which recognizes and cleaves the amino acid sequence GVVQASCRLA (SEG ID. No. 9).

The specific amino acid sequence of the specificity mediating peptide is selected by the skilled person depending on the intended specificity of the compound according to the invention for a particular tumor or a particular virus infection.

The units E1 and E2 as well as E2 and E3 are bound to each other, e.g. linear E1-E2-E3, NH₂-E1-E2-E3-COOH or COOH-E1-E2-E3-NH₂. This coupling can be realized by any manner, preferably by means of a covalent binding or a peptide bond between the units.

Surprisingly, the inventor was able to provide such a compound which inhibits itself in its capability to penetrate the cytoplasm or the cell nuclei of healthy cells. This self-inhibition is enabled by the presence of the at least two complexing agents for metals, namely as a part of E1 and a part of E3. Due to the size and configuration of the compound associated therewith, an uptake into the cytoplasm or the cell nucleus of healthy cells is prevented. The compound rather remains in the interstitium and is excreted from the organism after some time. However, in the neighborhood of tumor cells or virus-infected cells the specificity mediating peptide in E2 is recognized and cleaved by tumor- or virus-specific proteases respectively. As a result E3 is cleaved off from the compound and E1 can penetrate the cytoplasm and the cell nucleus of the tumor cell due to the transportation component. The separated unit E3 remains, after being cleaved off by the tumor or virus-specific protease, for a while in the interstitium and contributes, in the case of the provision of a contrast medium, to the signaling in the tumor or the infected area in an advantageous manner, until it is drained away from the interstitium and excreted from the organism. It is of particular advantage that the separated unit E3 has no neurotoxic properties as this is e.g. the case for the separated peptide fragments as described in WO 2006/056227 A1 (l.c.) and Jiang et al. (l.c.) or Liu et al. (l.c.).

The compound according to the invention is, in the neighborhood of tumor or virus-infected cells, so to say “activated” and cell membrane and cell nucleus membrane permeable, whereas such an activation in the presence of healthy cells is not taken place. This process is highly selective and specific so that the compound according to the invention displays its properties exclusively in tumor cells or virus-infected cells, respectively.

The compound according to the invention is surprisingly compact and small and consequently producible in a large scale for a reasonable price. Remarkably, the compound according to the invention only needs one transportation component which both mediates the permeability through the cell membrane as well as through the cell nucleus membrane. A separate TPU, such as in the compound known from WO 2004/050698, is not necessary. Also no nuclear localization sequence (NLS) is compellingly necessary. For a transportation component a short positively charged peptide is sufficient, e.g. PKKKRKV (SEQ ID No. 1) which, facultatively, can be extended by 4 arginine residues, i.e. PKKKRKVRRRR (SEQ ID No. 3). It goes without saying that variations thereof are conceivable.

In comparison to the compound known from WO 2004/050698 the compound according to the invention also does not need a bisulfite bridge. The compound according to the invention is, therefore, very stable and there is not the risk that it is cleaved and inactivated already in the blood stream.

As the inventors have realized the compound according to the invention does not have cytotoxicity to healthy cells, i.e. the vitality of the cells remains unaffected. However, in the cell nucleus of the tumor or virus-infected cells the cleavage product comprising E1 of the compound according to the invention initiates apoptosis without the occurrence of inflammatory processes or damaging of the surrounding healthy tissue. The cleavage product comprising E1 is then disposed of the body together with apoptotic tissue by macrophages within the context of the apoptotic process, so that in the case of the complexing of metals no toxic metals remain in the body, if applicable. The underlying mechanism initiating apoptosis is so far largely unknown, whereas the complexing agent and the transportation peptide may have an important function in this context. The compound according to the invention is therefore especially suitable as a therapeutic and diagnostic agent.

According to the invention it is preferred if the transportation component is a peptide (first transportation peptide).

Transportation peptides which mediate a permeability through the cell membrane and the nuclear cell membrane are comprehensively described in the prior art; cf. WO 2006/056227 A1. Martin M. E. and Rice K. G., in particular table 1 (l.c.) and Schwartz, J. J. and Zhang S., in particular table 2 (l.c.); the content of these documents is incorporated herein by reference. The transportation peptides are characterized by their positive net charge which is provided by an excess of positively charged amino acids, e.g. by arginine, lysine and histidine, and have a length of 2 to 40 amino acids, preferably of 3 to 20 amino acids, further preferred of 5 to 15 amino acids. A specific, concrete sequence of the amino acids is not necessary, whereas it is of an advantage if the transportation peptide comprises different positively charged amino acids. Decisive is the presence a positive net charge. The nuclear localization sequences (NLS) also belong to the transportation peptides. Transportation peptides are preferably linear but can also be branched. An appropriate transportation peptide comprises e.g. the following amino acid sequence: PKKKRKVRRRR (SEQ ID No. 3).

According to the invention it is preferred if the compound comprises a molecular weight which is about 2,000 kDa to about 10,000 kDa, preferably of about 3,000 kDa to ca. 6,000 kDa, highly preferred of about 4,000 kDa.

In this context it is of particular advantage that the compound according to the invention, due to the favorable ratio of the signalling component which is formed by the complexing agent after a metal is complexed, and the rest of the compound, emits a particular strong signal. The compound according to the invention is, therefore, able to emit a particularly strong signal and to display a particularly strong therapeutic effect.

The complexing agent for metals is preferably tetraazacyclododecane tetraacetic acid (DOTA).

DOTA is a complexing agent for gadolinium. It is characterized by its particular high stability and is, in vivo as well as in vitro considerably more stable than DTPA, the common complexing agent for gadolinium; cf. Magerstadt et al. (1986), An Alternative to Gd(DTPA) as a T1,2 Relaxation Agent for NMR Imaging or Spectroscopy, Magn. Reson. Med. 3(5): 808-12; Bousquet et al. (1988), Gd-DOTA: Characterization of a New Paramagnetic Complex, Radiology 166(3): 693-8.

According to a preferred development of the compound according to the invention the complexing agents for metals have a complexed gadolinium (Gd).

The use of gadolinium (Gd or Gd³⁺) has been proven of value in the magnetic resonance tomography. Gadolinium comprises a large ion capture radius and is, therefore, properly suitable for the neutron capture therapy (NCT). An overview on the characteristics and possibilities of use of this therapy can be found in Sauerwein W. (1993), Principles and History of Neutron Capture Therapy, Strahlenther. Onkol. 169: 1-6. Further, it recently turned out that gadolinium is X-ray dense and is, therefore, suited to be used in the computer tomography; cf. Henson et al. (2004), Gadolinium-enhanced CT Angiography of the Circle of Willis and Neck; AJNR Am. J. Neuroradiol. 25(6) 969-972. In addition, in contrast to e.g. iodine gadolinium does not cause allergies in the organism. In the following DOTA which has chelated Gd³⁺, e.g. Gd³⁺-DOTA, is referred to as Gd-DOTA or GdDOTA respectively.

After the specific uptake of the compound according to the invention into the tumor or virus-infected cells the organism is irradiated by a neutron radiation source which results in a conversion or activation of the neutron-absorbing compound according to the invention into a radiotoxic substance. Since the compound according to the invention is in direct contact to the DNA exclusively of transformed or virus-infected cells only such cells are destroyed in a targeted manner and side effects are largely avoided.

According to a preferred development the complexed gadolinium is radioactively labeled.

The labeling is realized e.g. in using radioactive gadolinium ¹⁵³Gd. Alternatively or additionally also ³²P, ³³P, ³⁵S, ³⁵Cl, ³⁷Cl, ¹⁵N, ¹³N, ¹³C, ¹⁴C, ²H, ³H, ¹²⁵I, ¹³¹I, ¹⁸F, ¹⁵O, ⁶⁷Ga, ¹¹¹In, etc. can be used. This measure has the advantage that the developed compound according to the invention can also be used in the radiotherapy and radiodiagnostics.

Appropriate fluorescent markers comprise fluorescein isothiocyanate (FITC) which is preferred, but also rhodamine, dansylchlorid, fluorescamine, green fluorescent protein (GFP), ethidiumbromide, 4′-6-diamidino-2-phenylindol (DAPI), coumarine, luciferase, phycoerythrine (PE), Cy2, Cy3.5, Cy5, Cy7, texas red, alexa fluor, fluor X, red 613, BODIPY-FL, TRITC, DS red, GFP, DS red, etc. By this measure the compound according to the invention is developed as a research tool which can be used in cell biology, e.g. to examine mechanisms of the cytoplasmatic or nuclear import of molecules in vitro or even in vivo. This marker enables the determination of the localization of the compound according to the invention by means of methods which are well established in the art, such as fluorescence microscopy which can even be used during a surgery in vivo or the near-infrared imaging for superficial tumors. In this case the markers in E1, E2, E3 can be different so that e.g. the interstitium (E3; marker 1, e.g. FITC) can be visualized by a color different than those to be used for the visualisation of the nucleus of the tumor cells (E1; marker 2, e.g. rhodamine).

According to a preferred development of the compound according to the invention E3 further comprises a second transportation peptide.

This measure has the advantage that after the cleavage of E2 by a tumor cell or virus-specific protease not only E1 but also E3 can be used for the intranuclear imaging and/or therapy. After the cleavage in E2 both cleavage products, i.e. E1 and E3, comprise a complexing agent for metals and a transportation peptide, so that both components can penetrate the cell membrane and the cell nuclear membrane.

In this context it is preferred if the first and/or second transportation peptide(s) comprise(s) a nuclear localisation sequence (NLS).

This measure has the advantage that already such transportation peptides are provided which have been proven as being functional. Examples of appropriate NLS are: PPKKKRKV (SEQ ID No. 10), and PKKKRKV (SEQ ID No. 1), both from the SV40-T-antigene. Further examples are KRRRER (SEQ ID No. 11) and KARKRLK (SEQ ID No. 12) from the simian cytomegalovirus. Further appropriate NLS originate from transcription factors:

| | | |
|---|---|---|
| NF-kappaB: | VQRKRQKLMP | (SEQ ID No. 13) |
| TFIIE-β: | SKKKKTKV | (SEQ ID No. 14) |
| Oct-6: | GRKRKKRT | (SEQ ID No. 15) |
| TCF-1-α: | GKKKKRKREKL | (SEQ ID No. 16) |
| HATF-3: | ERKKRRRE | (SEQ ID No. 17) |
| C. elegans SDC₃: | FKKFRKF | (SEQ ID No. 18) |

Another appropriate bipartite NLS is apoptin which contains two NLS sequences which can, however, also be used separately: KPPSKKR (SEQ ID No. 19) and RPRTAKRRIRL (SEQ ID No. 20).

An overview on NLS sequences can be found in Jans D. A. (1995), The Regulation of Protein Transport to the Nucleus by Phosphorylation, Biochem. J., 311(Pt 3), 705-716. The content of the before-identified publication is incorporated herein by reference.

Transportation peptides with sequences which comprise a homology of 80%, 85%, 90%, 95%, 98% with the before-identified sequences are also appropriate to mediate cell membrane and cell nuclear membrane permeability. This includes also so-called “mutated” NLS with an amended amino acid sequence and such having an amino acid, e.g. lysine, replaced against another amino acid, e.g. threonine.

According to a preferred configuration the first and/or second transportation peptide(s) comprise(s) a positive net charge.

The inventor has realized that such a transportation peptide is sufficient to ensure the cell wall and nuclear wall permeability of the compound according to the invention. In this context it is not necessary that a complete nuclear localization sequence (NLS) is provided. The transportation peptide consists in this case predominantly of basic amino acids, such as arginine, lysine, histidine or modified variations hereof.

In this context it is preferred if the first and/or second transportation peptides comprise a length of 3 to 20, further preferred of 5 to 15 and highly preferred of 7 amino acids.

This measure has the advantage that transportation peptides with a sufficient length are provided, however without unnecessarily enlarge the compound according to the invention. Examples of suitable transportation peptides which do not comprise motives of an NLS are the following: RRRKRRR (SEQ ID No. 21), RQIKIWFQNRRMKWKK (SEQ ID NO. 22), RAhxRahxRAhxRAhxRAhx (SEQ ID No. 23) (Ahx=amino hexanoic acid), YGRKKRQRRRP (SEQ ID No. 25), RKHRKH (SEQ ID No. 26) etc.

Further it is preferred if the first and/or second transportation peptide(s) comprise(s) the following amino acid sequence: PKKKRKV (SEQ ID No. 1).

This measure has the advantage that an NLS derived from the SV40-T-antigene is used which has been proven as particularly suitable. Transportation peptides having sequences which comprise a homology of 80%, 85%, 90%, 95%, 98% of the SEQ ID No. 1 are also suitable.

It is further preferred if the first and/or second transportation peptide(s) of the compound according to the invention comprise(s) the following amino acid sequence: RRRR (SEQ ID No. 2).

The inventor has surprisingly realized that such a short transportation peptide which only consists of 4 arginine residues is sufficient to ensure the cell membrane and cell nuclear membrane permeability of the compound. Transportation peptides having sequences which comprise a homology of 80%, 85%, 90%, 95%, 98% of the SEQ ID No. 2 are also suitable.

It is particularly preferred if the first and/or second transportation peptide(s) of the compound according to the invention comprise(s) the following amino acid sequence: PKKKRKVRRRR (SEQ ID No. 3).

The inventor has realized that by the terminal addition of 4 arginine residues to the NLS derived from the SV40-T-antigene the transportation properties can be considerably improved. Transportation peptides with sequences which comprise a homology of 80%, 85%, 90%, 95%, 98% to the SEQ ID No. 3 are also suitable.

According to a preferred configuration the specificity mediating peptide of the compound according to the invention comprises a length of 2 to 20, preferably of 3 to 15 and highly preferably of 5 amino acids.

This measure has the advantage that a peptide having such a length is provided which is sufficient for the mediation of the specificity, however without unnecessarily enlarge the compound according to the invention. The indicated length has been proven in this context as being particularly suitable.

According to a preferred configuration the specificity mediating peptide comprises the following amino acid sequence: PLGLA (SEQ ID No. 4) or PLGVR (SEQ ID No. 5).

This measure has the advantage that such a compound is provided by which specifically and highly selectively brain tumors can be imaged or treated, respectively. The indicated amino acid sequence is recognized and cleaved by the matrix metalloproteinase 2 (MMP-2) which is characteristic for brain tumors. Specificity mediating peptides with sequences comprising a homology of 80%, 85%, 90%, 95%, 98% with the SEQ ID Nos. 4 or 5 are also suitable.

According to a preferred development of the compound according to the invention the complexing agent for metals and/or the fluorescent marker is covalently bound to the ε-amino group of a lysine residue.

By this measure the constructive conditions for a stable attachment of the complexing agent are provided. As a result, e.g. DOTA or FITC can be coupled by simple routine measures to the ε-amino group of such a lysine residue which is part of E1 and/or E2. DOTA and FITC can also simultaneously be bound to a lysine residue, in particular if the lysine residue is positioned at the N-terminus of the compound. The lysine residue can also be a part of the transportation peptide but can also follow the N- or C-termini thereof and can also represent the connecting member to E2. The lysine residue can, in this context, preferably be covalently bound via its α-amino group or its α-carboxyl group to the transportation peptide or to E2. The lysine residue is, so to say, a “hanger” for the complexing agent or the fluorescent marker, respectively.

According to a preferred configuration of the compound according to the invention E1 and/or E3 comprises a spacer which preferably comprises two amino acids, which spaces the complexing agent from the fluorescence marker.

The spacer has the function to prevent any steric interferences between the complexing agent and the fluorescent marker. This measure has the advantage that the complexing and signaling capacity of the complexing agent and the fluorescent marker is guaranteed or even increased. The spacer can consist of a short peptide of any 2 to 15 amino acids, e.g. of 2 lysine residues, whereas, however, 2 glycine residues are preferred. Alternatively the spacer can be formed by amino hexanoic acid (Ahx), beta-alanine, arginine residues etc.

A further subject matter of the present invention relates to the use of the compound according to the invention for manufacturing a diagnostic and/or therapeutic composition which is preferably a contrast medium for the magnetic resonance tomography (MRT) or for the nuclear medicine.

Another subject matter of the present invention relates to a diagnostic and therapeutic composition which comprises a diagnostically or therapeutically accept-able carrier.

Diagnostically and therapeutically acceptable carriers are comprehensively described in the prior art and are selected by the skilled person according to the intended form of use; c.f. Bauer et al. (1999), Lehrbuch der pharmazeutischen Technologie, 6th edition, Wissenschaftliche Verlagsgesellschaft mbH Stuttgart 1999; Row R. C., Sheskey et al. (2006), Handbook of Pharmaceutical Excipients, 5th edition, Pharmaceutical Press and American Pharmacists Association. The content of both of the before-identified publications is incorporated herein by reference.

Another subject matter of the present invention relates to a method for the diagnostic and/or therapeutic treatment of a living being, which comprises the following steps: (a) administration of the diagnostic and/or therapeutic composition according to the invention into the living being, and (b) performance of an imaging method.

The imaging method refers to magnetic resonance tomography (MRT), (auto)radiography, PET, scintigraphy, computer tomography, etc.

It goes without saying that the before-mentioned features and those to be explained in the following cannot only be used in the combinations indicated in each case, but also in other combinations or alone, without departing from the scope of the present invention.

The present invention is now explained in more detail which results in more characteristics and advantages of the invention. Reference is made to the enclosed figures.

### BRIEF DESCRIPTION OF THE DRAWINGS

FIG. 1 schematically shows different configurations of the compound according to the invention;

FIG. 2 schematically shows the active principle of the compound according to the invention;

FIG. 3 shows the result of a confocal laser microscopy on LN18 glioma cells after the incubation of the compound V4 in presence of inhibited inactivated matrix metallo-proteinase 2 (MMP-2) or activated MMP-2. A staining of the cell nuclei can only be observed after the cleavage of the conjugate by the active MMP-2;

FIG. 4 shows the result of a confocal laser microscopy on U373 (top) and L18 (below) glioma cells after the incubation with the compound V5 in culture medium with inhibited inactivated MMP-2 or active MMP-2. Only with conjugate V5 cleaved by the active MMP-2 the conjugate accumulates in the cell nuclei (below). If the compound V5 is not cleaved, the cells remain unstained;

FIG. 5 shows the result of a FACS (fluorescence activated cell sorting) analysis of LN18 glioma cells after the incubation with the compound V2 or V4 in culture medium each with inactive or active MMP-2. If both conjugates are cleaved by the active MMP-2 a considerable increase of the strongly stained cells can be observed, which can be seen by a displacement of the peak of the histogram to the right. The displacement is more pronounced after the cleavage of the compound V4;

FIG. 6 shows the result of a HPLC (high pressure liquid chromatography) of the compounds V5 or V5a, respectively, i.e. with or without gadolinium, before and after the cleavage by MMP-2. The large peak before the cleavage has been split into two new peaks which represent both of the cleavage products;

FIG. 7 shows the result of a magnetic resonance relaxometry on LN18 glioma cell centrifugates after the incubation with the compounds V2a and V4a (in each case 26 and 130 μm in a medium with active MMP-2 or inactive MMP-2). The incubation with active MMP-2 (left row) results in a considerably higher signal intensity at TR: 400 ms as the incubation with inactive MMP-2 (right column). After the incubation of the cells they were washed and centrifuged for three times;

FIG. 8 shows a magnetic resonance tomography image of a human NL18 glioma between the anterior horns and the lateral ventricles of a nude mouse. 30 minutes after the intraperitoneal administration of the compound V4a the intensity of the signal in the tumor considerably decreases in comparison to the native image.

### DESCRIPTION OF PREFERRED EMBODIMENTS

### Example 1

### Principle Design of the Compound According to the Invention

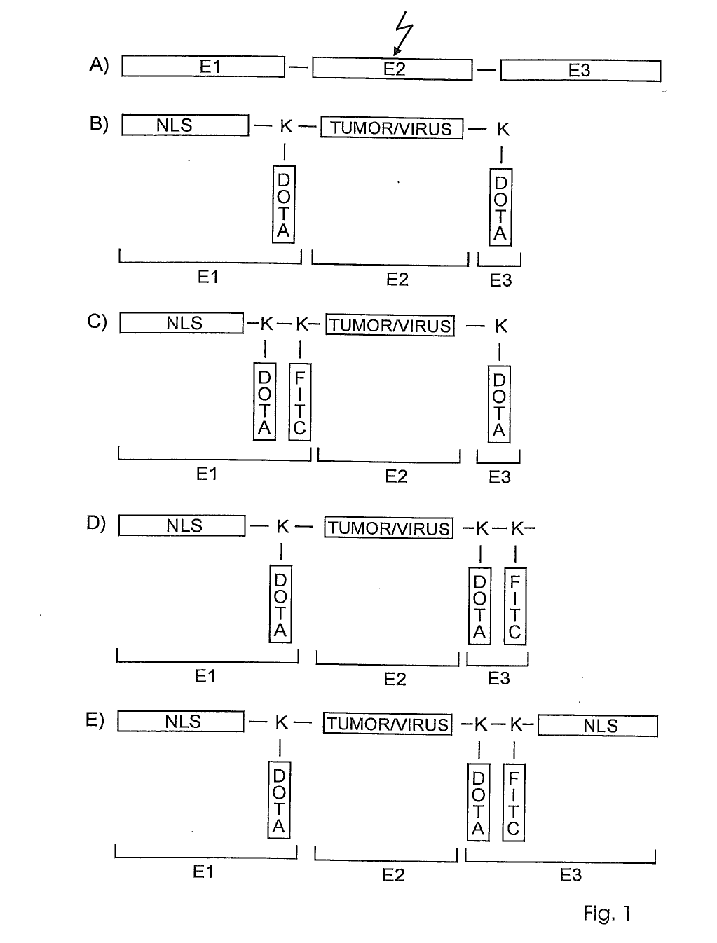

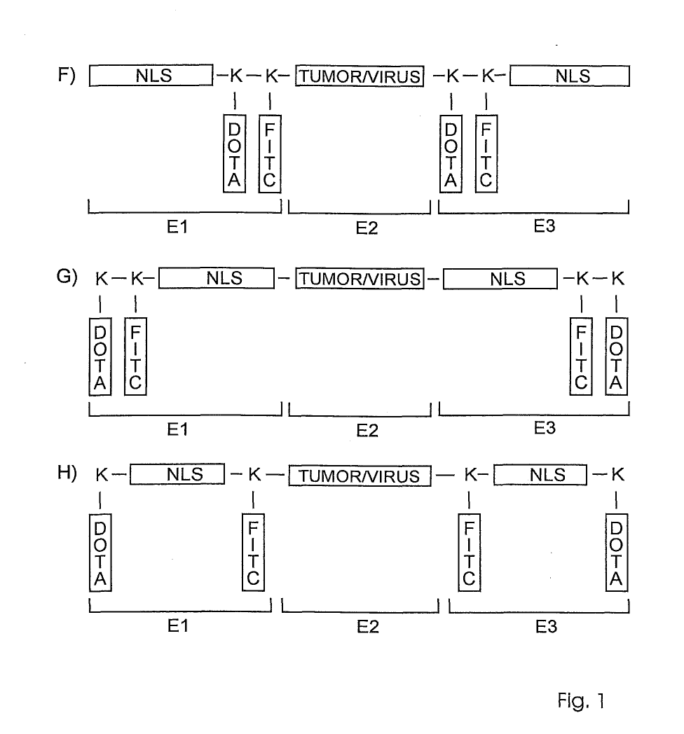

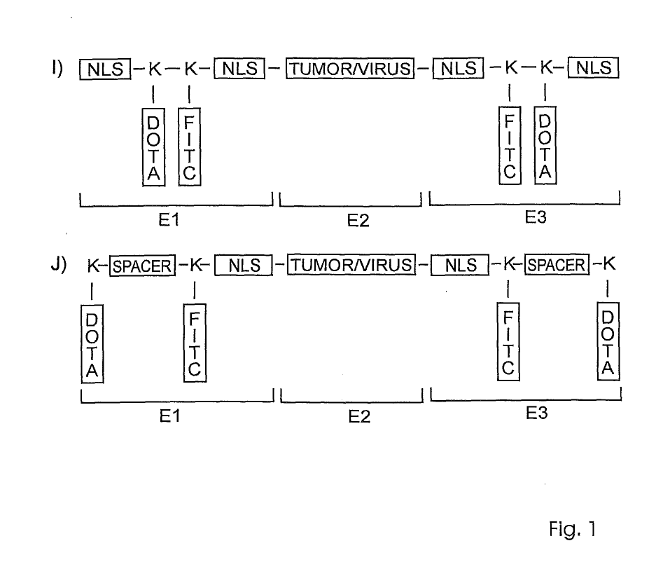

In FIG. 1 different embodiments of the compound according to the invention are shown in a schematic manner.

Partial FIG. A shows the basic structure of the compound in linear form. On the left side the N-terminus and on the right side the C-terminus of a peptidic compound according to the invention is shown. The first unit E1 which comprises the transportation peptide and the complexing agent for metals, is preferably covalently linked to the second unit E2 which comprises the cleavage side for the tumor or virus-specific proteases, i.e. the specificity mediating peptide. The folded arrow symbolizes the working or cleavage side for the tumor or virus-specific proteases in E2. The second unit E2 is preferably covalently bound to the third unit E3 which comprises the second complexing agent for metals.

In the partial FIG. B a preferred configuration of the compound according to the invention is shown in more detail. The transportation peptide is represented by a nuclear localization sequence (NLS) which is, at its C-terminal end followed by a lysine residue (K), which is, via its α-amino group, covalently bound to the NLS. Via the ε-amino group of the lysine residue a covalent coupling of the complexing agent occurs, which is represented by DOTA. The lysine residue serves, so to say, as a “hanger” for DOTA. In this embodiment the NLS, the lysine residue and DOTA are attributed to the first unit E1. The covalent binding of the specificity mediating peptide (TUMOR/VIRUS) which comprises a cleavage site for tumor or virus-specific proteases occurs via the α-carboxyl group of the lysine residue. The specificity mediating peptide represents the second unit E2. The C-terminus of the specificity mediating peptide is linked, via a peptide bond, to the α-amino acid of a further lysine residue which, in this embodiment, is attributed to the third unit E3, the ε-amino group of which is covalently bound to the second complexing agent which is also represented by DOTA.

In the partial FIG. C the first unit E1 comprises a further lysine residue (K) which follows the first lysine residue of the C-terminus by means of a peptide bond. The ε-amino group of the second lysine residue is covalently bound to a fluorescent marker which is represented by FITC.

In the embodiment of the partial FIG. D the fluorescent marker which is coupled via a lysine residue is located at the C-terminus of the compound according to the invention, i.e. in the third unit E3.

In the embodiment of the compound according to the invention in figure E the third unit E3 comprises a further nuclear localization sequence (NLS) which is located at the C-terminus of the compound according to the invention and is, with its N-terminus, covalently coupled to the lysine residue which carries as a “hanger” the fluorescence marker FITC.

In a particular configuration of the compound according to the invention in partial FIG. F not only the first unit E1 or the third unit E3 comprises the fluorescence marker FITC beside the complexing agent DOTA, but the first unit E1 and also the third unit E3. The fluorescence signal coming from the compound according to the invention is herewith increased.

In the configuration of the compound according to the invention in partial FIG. G a modification with regard to the configuration in FIG. F has been done insofar as the complexing agents and the fluorescence marker as a part of the first unit E1 and the third unit E3 are now located at the N- or C-terminus of the compound. The NLS of E1 is consequently covalently linked to the specificity mediating peptide of E2 by its C-terminus, and the NLS of E3 is covalently linked to the specificity mediating peptide of E2 by its N-terminus.

The configuration according to partial FIG. H comprises in the first unit E1 and the third unit E3 the NLS flanked by the complexing agent DOTA and the fluorescence marker FITC.

In the configuration in partial FIG. 1 the first unit E1 and the third unit E3 each comprise two NLS, where the complexing agent DOTA and FITC are located in between.

Partial FIG. J shows a particular configuration of the compound according to the invention where between the complexing agent DOTA and the fluorescence marker FITC a so called spacer (SPACER) is located, which can consist of two amino acids, preferably of two glycine residues. The spacer spaces the complexing agent from the fluorescence marker and, as a result, prevents negatively effecting interactions between the two components and ensures their functionality.

The particular configurations of the compound according to the invention in partial FIGS. E to J have the advantage that after the cleavage of the compound in E2 both cleavage products, i.e. E1 and E3, can penetrate the cytoplasm and the cell nucleus of tumor cells, due to the respective presence of a transportation peptide or a nuclear localization sequence, and there display their effect. In the particular configuration of the compound according to the invention in partial FIGS. A to D this can only be done by the cleavage product E1, since only this comprises a transportation peptide or an NLS, respectively. The cleavage product E3 remains in the interstitium and is excreted from the interstitium and the organism after a certain time.

It goes without saying that the indicated configurations of the compounds are examples which are not to be understood as being limiting. The individual components within one unit can of course vary in their localization and position. Furthermore, combinations of the different configurations are possible as long as the principle assembly, as shown in partial FIG. A, is maintained.

### Example 2

### Principle of Operation of the Compound According to the Invention

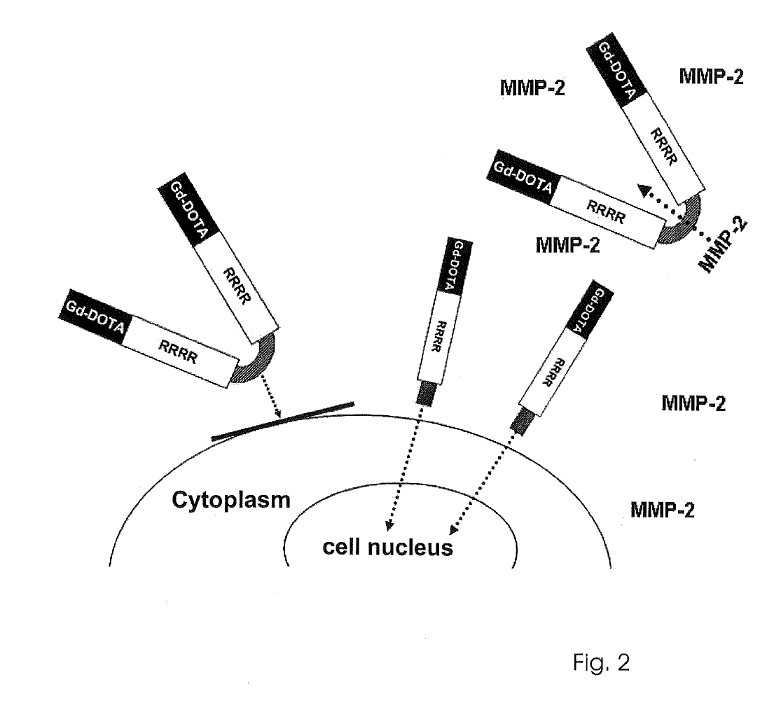

In FIG. 2 the principle of operation is schematically explained by means of a particular configuration of the compound according to the invention, where the first unit E1 as well as the third unit E3 comprise a transportation peptide which is represented by 4 arginine residues (RRRR). At the C- or N-terminal end of the component according to the invention, a complexing agent is covalently linked, which has a chelated gadolinium (Gd-DOTA). In the middle or the center of the compound according to the invention, the unit E2 is located, which comprises a cleavage site, which is recognized and cleaved by the tumor specific protease MMP-2.

This mirror-inverted assembled compound according to the invention cannot enter healthy non-transformed cells due to its size and the lack of MMP-2 (left). Only in presence of transformed tumor cells which secrete MMP-2 into their environment, the specificity mediating centrally located peptide is cleaved. The released cleavage product which contains E1 and E3 as well as fragments of E2, can in each case be uptaken into the cytoplasm and the cell nucleus of the tumor cell due to the arginine-rich transportation peptide and their reduced sizes.

After the induction of the apoptosis the cleavage products are “disposed” via macrophages and any complexed metal is finally excreted from the organism.

### Example 3

### Material and Methods

### 3.1 Synthesis of the Compound According to the Invention

The synthesis occurs according to the Fmoc solid phase method on an Eppendorf ECOSYN P peptide synthesizer (Eppendorf-Biotronik, Hamburg, Germany). The 9-fluorenylmethyloxycarbonyl group cleavable under basic conditions, was used as an amino protective group. As a carrier material the tentagel S rink-amid resin (Rapp-Polymere, Tubingen, Germany) was used. The syntheses were performed in a 0.1 mMol scale. The couplings were performed with the protected Fmoc-amino acids with a 4-fold excess of 2 (1H Benzotriaol-1-yl)-1.1.3.3-tetramethyluronium tetrafluoroborate [TBTU] (4 eq) in the presence of 8 eq. diisopropylethylamine within 40 minutes. As a protective group for the side chains were used: For lysine: Tert. Butyloxycarbonyl (Boc), for arginine: pbf (N⁶-2.2.3.6.7-pentamethyl-dihydrobenzofuran-5-sulfonyl).

For the side chains which were provided with DOTA, a lysine derivative with 4-methoxytrityl (Mmt)-side chain protection was used, for the positions which should carry the fluorescence urea residue the Lys-Dde-derivative (Dde=1-(4,4-dimethyl-2,6-dioxocyclohex-1-ylidene) was used. After the coupling the Fmoc residue was in each case cleaved off with 25% piperidin/DMF-solution in 11 minutes.

After several washes with dimethylformamide (DMF) the peptide resin was prepared for an additional coupling.

After a successive assembly of the peptide starting from the C-terminus the N-terminal amino acid is, in the case of proline, introduced into the peptide as Boc-proline.

In all other cases after the Fmoc-group has been cleaved off the peptide is protected by the Boc-group. This is achieved by shaking the peptide resin with 20 eq. di-tert-butyldicarbonate [Boc2O]/10 eq. diisopropylethylamine in dichloromethane within one hour at room temperature.

Then the mmt side chain protective group is cleaved off within one hour by several additions of 1% TFA/DCM-solution containing 1% triisopropylsilane. After several washes with DMF and neutralizing the resulting TFA-salts with diisopropylethanamine the exposed side chain is available for a coupling with 1,4,7,10-tetraazacyclododecane-1.4.7.tritert-butylester-10-acetic acid (DOTA) in each case 3 eq. in presence of 3 eq. TBTU and 6 eq. diisopropylethylamine within 1.5 h at room temperature. Then the dde protective group is cleaved off by several additions of a 2.5% hydracine hydrate solution in DMF to the resin within one hour.

After several washes with DMF the fluorescein urea derivative is coupled via 0.5 mM fluorescein 5(6)-isothiocyanate in the presence of the eq. amount diisopropylethylamine in DMSO over night at room temperature.

After several washes with DMF, methanol and dichloromethane after the drying the remaining protective groups and the peptide are cleaved off from the resin simultaneously; this occurs by an agitation of the dried resin in a mixture of 12 ml TFA, 0.3 ml Ethandithiol (EDT), 0.3 ml Anisol, 0.3 ml water and 0.1 ml triisopropylsilane for three hours at room temperature.

Then it is directly filtered in cooled, absolute diethylether. The precipitated peptide is filtered, washed with ether and dried in vacuum. The obtained raw peptides are purified as in part I by means of a semi-preparative HPLC. The DOTA-peptides are subsequently added to the eq. amount of gadoliniumchlorid solution, brought to a pH-value of 5.2-5.6 with 0.1 m NaOH and are agitated for 5 hours at 50° C.

After the addition of several drops of acetic acid the solution is lyophilized. The analytics is performed by an analytic HPLC and mass spectrometry (purity at least 98%).

By this manner the following compounds were synthesized:

TABLE 1
Synthesized compounds

| Comp.# | Formula | MW [kDa] |
|---|---|---|
| V1 | PKKKRKV-K(FITC)-GG-K(DOTA) | |
| V2 | PKKKRKV-K(FITC)-GG-K(DOTA)-PLGVR-K | ≈3.000 |
| | (DOTA) | |
| V3 | PKKKRKVRRR-K(FITC)-GG-(K(DOTA) | |
| V4 | K(DOTA)-G-PLGLA-GGPKKKRKVRRRR-K | ≈3.800 |
| | (FITC)-GG-K(DOTA) | |
| V5 | K(DOTA)-GG-K(FITC)-RRRR-G-PLGLA-G- | ≈4.100 |
| | RRRR-K(FITC)-GG-K(DOTA) | |
| V1A | PKKKRKV-K(FITC)-GG-K(GdDOTA) | |
| V2A | PKKKRKV-K(FITC)-GG-K(GdDOTA)-PLGVR-K | ≈3.400 |
| | (GdDOTA) | |
| V3A | PKKKRKVRRRR-K(FITC)-GG-K(GdDOTA) | |
| V4A | K-(GdDOTA)-G-PLGLA-GG-PKKKRKVRRRR-K | ≈4.100 |
| | (FITC)-GG-K(GdDOTA) | |
| V5A | K-(GdDOTA)-GG-K(FITC)-RRRR-G-PLGLA- | ≈4.450 |
| | G-RRRR-K(FITC)-GG-K(GdDOTA) | |

One letter amino acid code: K, lysine; r; arginine; P, proline; V, valine; G, glycine; FITC: fluorescent dye, fluorescein isothiocyanate; DOTA: complexing agent for gadolinium, tetraazacyclododecane tetraacetic acid; Gd: gadolinium or gadoliniumion Gd³⁺.

The transportation peptide is underlined, the specificity mediating peptide is shown in italic, the spacer is shown in bold letters. The N-terminus is on the left, the C-terminus on the right.

### 3.2 Cleavage Test

The compounds 1, 4 and 5 or 2A, 4A and 5A, respectively, were in each case dissolved in HEPES buffer (μM), wherein one was already containing active MMP-2 (calbiochem), and the other was already containing the inactive proform of MMP-2 (calbiochem).

The incubation in HEPES buffer with active MMP-2 was made for 2 hours.

For the conversion of the inactive proform into the active MMP-2 APMA (4-aminophenyl mercuric acetate) was used. For this 1% APMA stock solution (100 mM in DMSO) was added to the solution containing the MMP-2 proenzyme and the compounds (final APMA concentration: 1 mM, with 1% DMSO).

In the following also an incubation of 2 hours was performed. In addition the tests were made with the MMP-2 inhibitor I (Calbiochem). All six compounds were, as a control, also incubated into HEPES buffer without MMP-2 for two hours.

The cleavage products were examined by HPLC:

Column: Nucleosil 100 5 μm C₁₈ (250×4); buffer A: 0.07% CF₃COOH/H₂O; buffer B: 0.58% CF₃COOH/80% CH₃CN

10→90% B in 36 min; 170 bar; 1 ml/min; 214 nm

### 3.3 Confocal Laser Scanning Microscopy (CLSM) and Vitality/Apoptosis Analysis

Human malignant glioma cells (U373 and LN18) were seated into 25 cm² culture flasks which contained 3 ml RPMI medium. This was left as it is for one day so that enough matrix metalloprotenases (MMP-2) could accumulate. For the activation of the inactive proform of MMP-2 which was present in the medium, the latter was incubated shortly before the analysis with APMA (4-aminophenyl mercuric acetate) dissolved in 0.1% DMSO (confluence of the cells: 70%).

In a first test the compounds 2, 4 and 5 and the compounds 2A, 4A and 5A, in each case without MMP-2 inhibitor, were dissolved in the media of 12 flasks (130 μM) (both cell lines). In a second test the same compounds were again dissolved in the media of 12 flasks (130 μM), however now with MMP-2 inhibitor I (Calbiochem).

The blockage of the MMP-2 with MMP-2 inhibitor I was performed as previously described by Yin et al. (2006), Matrix Metallo-proteinases Expressed by Astrocytes Mediate Extracellular Amyloid-β Peptide Catabolism, The Journal of Neuro-science 26(43): 10939-10948. As a control four small flasks were used wherein both cell lines were incubated in a one day old medium with and also without APMA.

The detection of phosphatidylserine in the outer membrane leaflet for determining the apoptosis was performed by using annexin-V Alexa™ 568 reagent according to the recommendation of the manufacturer (Roche Molecular Biochemical, Indianapolis, USA).

For the confocal laser scanning microscopy an inverted LSM510 laser scanning microscope (Karl Zeiss, Jena, Deutschland) (Objectives: LD Achroplan 40×0.6, Plan Neofluar 20×0.50, 40×0.75) was used [fluorescence excitation at 488 nm (argon-ion laser) and 534 nm (helium-neon laser)]. Superimposed images of FITC- and alexa-stained cells were produced. All measurements were performed on living, non-fixed cells for three times.

### 3.4 Magnetic-Resonance Relaxometry

Human U373 and LN18 glioma cells were grown in 25 cm² culture flasks (70% confluence). Accutase™ (PAA laboratories, Pasching, Austria) was added to strip off the cells from the bottom of the culture flasks.

In a first try the cells were collected and subsequently distributed on 16 Eppendorf-vials (6×10⁶ cells per vial). The cells in the first four vials were used as a control (only RPMI medium with and also without APMA, two cell lines). The two cell lines in the other twelve vials were incubated with the compounds 2A, 4A and 5A (130 and 260 μM each) for 2 hours at 37° C. and 5% CO₂ with, but also without MMP-2 inhibitor I. In the following it was washed for three times with PBS and centrifuged with 800 rpm (rounds per minute) for 5 minutes.

In another test adherent U373 and LN18 glioma cells were incubated with the compounds 2A, 4A and 4A, and were stripped off in the following and collected in Eppendorf vials for the MRT. The MRT analysis of the Eppendorf vials with the cell centrifugates were performed in a 3 Tesla full body MRT apparatus (Trio, Siemens Magnetom Sonata, circular polarised knee coil).

The following spin echo sequence was used to obtain sagittal T1-weighted MRT images.

TR (repetition time): TE (echo time): 7.4 ms, flip angle 90°, averages: 1, concatenations: 2, measurements: 2, number of slices: 19, distance factor: 30%, slice thickness: 3 mm, field of view read: 180 mm, field of view phase: 100%, base resolution: 256, phase resolution: 100%, voxel size: 0.7×0.7×3.0 mm, scan time: 1:48 min.

By means of multiple spin echo measurements (TR: 20-8000 ms, 50 different TR values) different signal intensities were measured through which the T1 relaxation time could be determined.

TR: 20-8000 ms (50 different TR values), TE: 6.4 ms, flip angle 90°, averages: 1, measurements; 1, number of slices: 1, slice thickness: 1 mm, field of view read: 120 mm, field of view phase: 87.5, base resolution 128, phase resolution; 100% voxel size: 0.9×0.9×1 mm.

For the analysis and calculations a Matlab programme (Math Works, Natick, Mass., USA) was used. The T1 values were determined via a two-parameter fit. All signal curves were examined and rated as being mono-exponential. All tests were performed three times.

### 3.5 FACS

The FACS analysis was performed on a Becton-Dickinson FACSCalibur. [100 ml of the cell suspension (1×10⁶ cells+300 ml FACS buffer (DPBS buffer with 1% paraformaldehyde)]. Approximately 25.000 to 35.000 cells were measured per sample [fluorescence excitation: argon-ion laser (480 nm), fluorescence detection: 540 to 565 nm band-path filter]. The tests were repeated two times.

### 3.6 Implantation of the Tumors

The animal experiments were approved by the Regional Board of Tubingen.

Female nude mice CD1(Nu/Nu) were obtained from Charles River (Sulzfeld, Germany) (weight: 25 g; age: 7 weeks). Human LN18 glioma cells were grown and implanted into the brain as described by Friese et al. (2003), MICA/NKG2D-mediated Immunogene Therapy of Experimental Gliomas, Cancer Res. 63(24): 8996-9006. The tests were performed 3 weeks after the implantation.

### 3.7 In Vivo Magnetic Resonance Tomography and Confocal Laser Scanning Microscopy

3.6 mg of the compound 4A were dissolved in 1 ml isotonic saline solution and intraperitoneally injected into 2 mice. Further, 3.6 mg of compound 4A were dissolved in 1 ml isotonic saline solution together with 0.5 mg MMP-2 inhibitor I, and in intraperitoneally injected into 2 mice. A mouse was used as control which were injected with 1 ml pure saline solution. Narcosis was performed with ketamine (100 mg/kg) and xylazine (10 mg/kg) intraperitoneally.

MRT was performed with a 3 Tesla full body apparatus (Trio. Siemens).

The mice were placed face down in a wrist array coil.

The MRT protocol consisted of the following sequences:

Tse 3D sequence: slice thickness 0.3125 mm, field of view read 63 mm, field of view phase 100.0%, base resolution 256, phase resolution 100%, slice resolution 100%, voxel seize 0.2×0.2×0.3 mm, slab group 1, slabs 1, slices per slab 16, TR 300 ms, TE 15 ms, flip angle 70, distance factor 50, scan time 12:02 min.

T1 weighted transverse images: slice thickness 2 mm, field of view read 31 mm, field of view phase 82.3%, voxel seize 0.2×0.2×2 mm, TR 600 ms, TE 18 ms, flip angle 180, number of slices 12, distance factor 0, scan time 11:35 min.

120 minutes after the intraperitoneal injection the organs including the brain were removed and frozen in Tissue-Tek OCT (Sakuta, Tokio, Japan) liquid nitro-gene.

The cell nuclei were, as a control, also stained with propidium iodide, a typical dye for the cell nuclei.

The organs including the brain and the tumor, were removed 120 minutes after the intraperitoneal injection and frozen into Tissue-Tek OCT and liquid nitro-gene.

For the definite localization of the cell nuclei they were stained with propidium iodide.

The conjugate was localized in the cells by means of a confocal laser scanning microscope.

Sections stained with haematoxylin and eosin (H&E) were prepared for the transmission light microscopy to be sure that a tumor was existing.

### 3.8 Semi-Thin Sections

A part of the cells examined by the FACS analysis were fixed by 2% Agar, dehydrogenated in ethanol, embedded into lowicryl K4M (Polysciences, Eppelheim, Germany) and polymerized at room temperature according to the recommendations of the manufacturer. Semi-thin sections (about 0.4 μm) were prepared and evaluated by the fluorescence microscope.

### Example 4

### Results

### 4.1 Components

The analyses were performed with the components V2, V4 and V5 without gadolinium and the corresponding gadolinium-containing compounds V2A, V4A and V5A. The NLS of the SV40-T-antigen alone with the DOTA complex (compound 2), which was linked at its C-terminal end via an MMP-2 sensitive peptide bridge to a second DOTA complex, and the NLS of the SV40-T-antigen which was extended by 4 arginines and was, at the N-terminal end, linked via an MMP-2 sensitive peptide bridge to a second DOTA complex (compound 4). In the compound 54 arginines and one FITC molecule and one DOTA complex each were inversely linked via an MMP-2 sensitive peptide bridge, so that in total 2 FITC molecules and 2 GdDOTA complexes were present in one compound; cf. also Tab. 1 and FIG. 2.

### 4.2 Tumor Specificity/Apoptosis

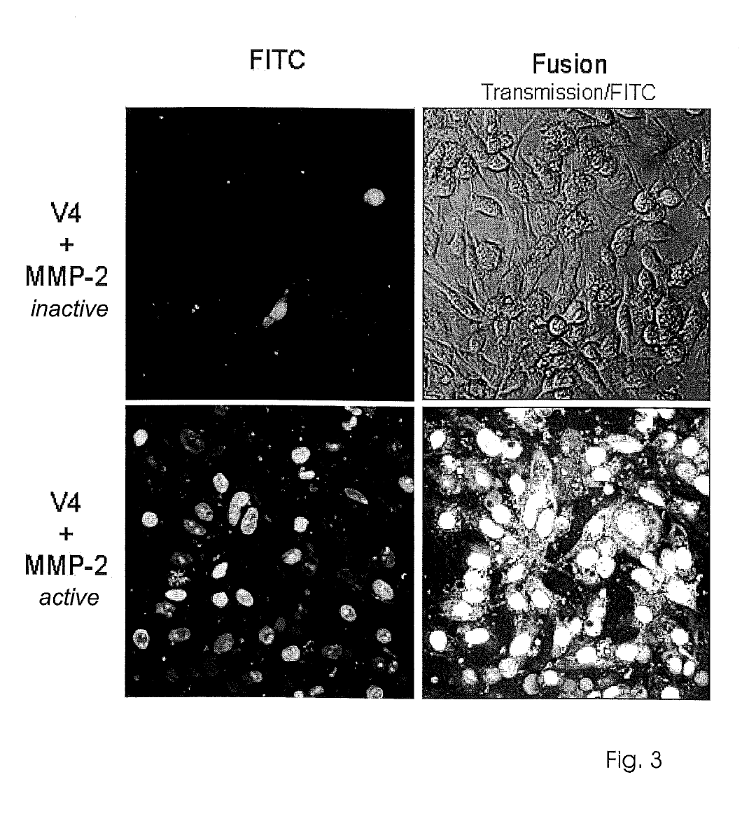

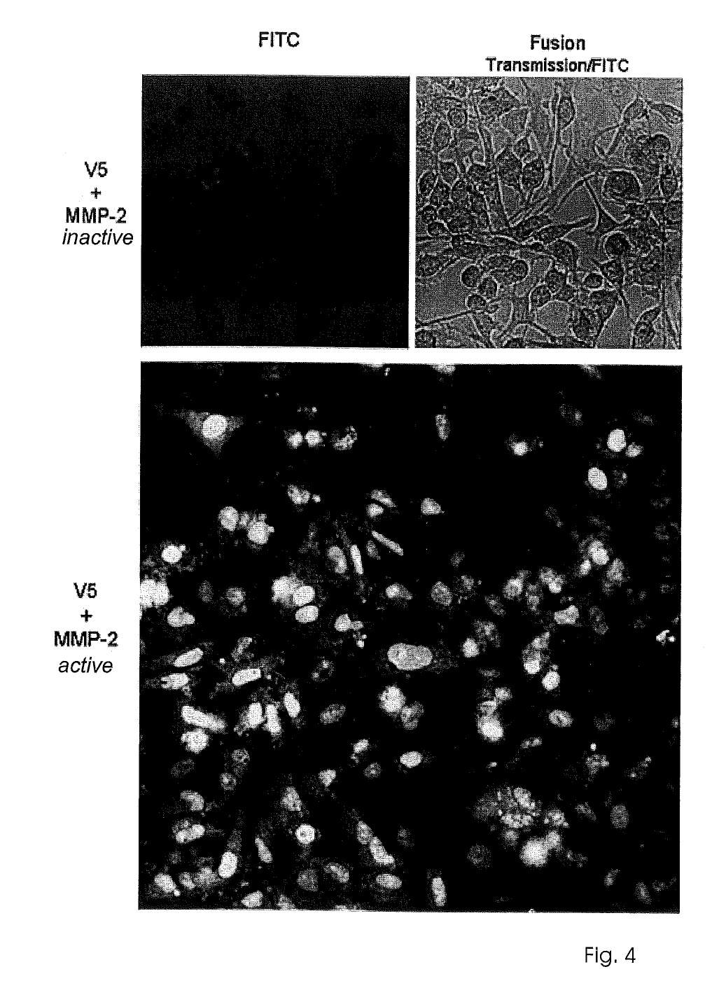

The human malignant LN18 and U373 glioma cells (adherent and stripped off) showed after an incubation in RPMI medium with APMA (4-aminophenyl mercuric acetate, activator of MMP-2) alone with and also without MMP-2 inhibitor I no autofluorescence in the confocal laser scanning microscopy; cf. FIGS. 3 to 5, each on the top.

Both the MMP-2 inhibitor I as well as APMA in the medium did not result in a damage of the cell vitality.

In the absence of the inhibitor in the APMA-containing medium the incubation of LN18 and U373 glioma cells (adherent and stripped off) with the conjugates 2, 4 and 5 (without gadolinium) and 2A, 4A and 5A (with gadolinium) (130 μM) did only result in few stained cell nuclei in the CLSM and FACS analysis; cf. FIGS. 3, 4 and 5—“MMP-2 inactive”. The cells did not show any signs of cell death (data not shown).

When the inhibitor was missing in the APMA-containing medium the incubation of LN18 and U373 glioma cells (adherent and stripped off) with the identical components (130 μM) result, however, in a massive increase of the staining of the cell nuclei in the CLSM and FACS analyses, whereas these cells were only apoptotic after an incubation with the compounds 4 and 4A as well as with the compounds 5 and 5A (binding of Alexa annexin to phosphatidyl serine, data not shown; morphologic alterations); cf. FIGS. 3, 4 and 5—“MMP-2 active”.

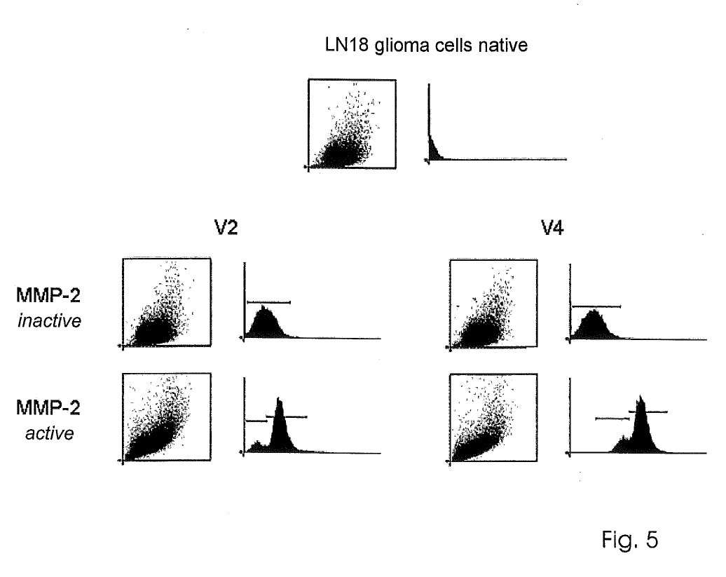

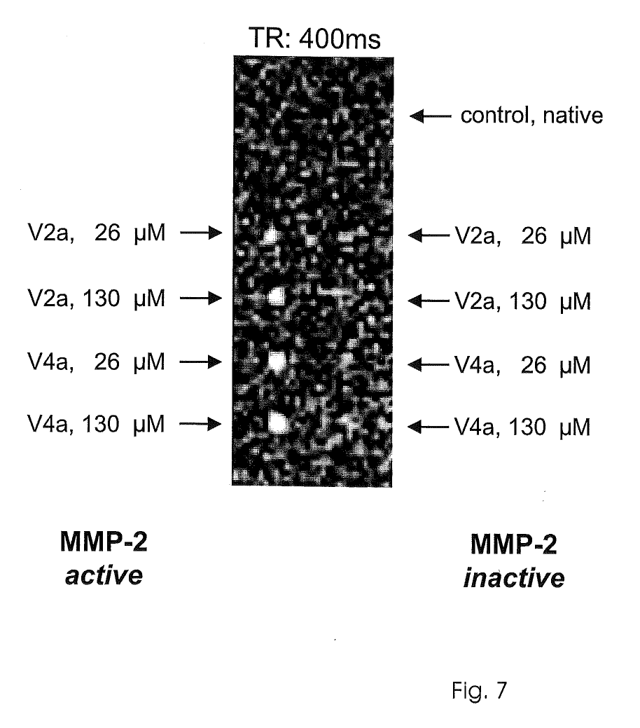

The gadolinium-containing compounds 4A and 5A and the gadolinium-free compounds 4 and 5 after their cleavage by MMP-2 in comparison to the components 2 and 2A result, depending on the concentration, in a stronger fluorescence (FIG. 5; exemplarily shown for compounds 2 and 4) and in a higher signal intensity in the MRT (cf. FIG. 7; exemplarily shown for compounds 2A and 4A), wherein the absence of gadolinium in the DOTA complexes in all compounds only result in a minor derogation of the transmembrane transport.

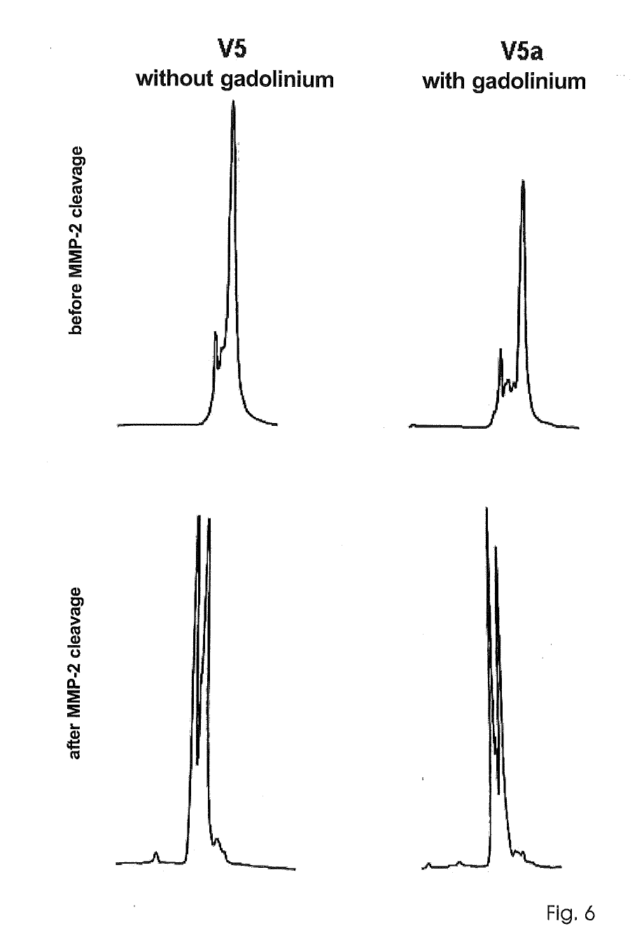

By means of the HPLC (chromatography) it could be shown that the compounds 2, 4 and 5 or 2A, 4A and 5A, respectively, were cleaved in the presence of the already active MMP-2 as well as the proform of MMP-2 activated by APMA (4-aminophenylmercuric acetate, activator of the MMP-2), and that this cleavage is prevented by the inhibitor; cf. FIG. 6 (exemplarily shown for compounds 5 and 5A).

In the MRT the cells showed after the incubation with inhibitor-containing medium only a minor shortening of the T1 time in comparison to the native control (26 and 130 μM) cf. FIG. 7 (exemplarily shown for compounds 2A and 4A). However, a more distinct shortening of the T1 time could be observed after the incubation of the cells with the components in an inhibitor-free medium, whereas the components 4A and 5A in comparison to component 2A showed a stronger shortening of the T1 time depending on the concentration; cf. FIG. 7 (exemplarily shown for compounds 2A and 4a).

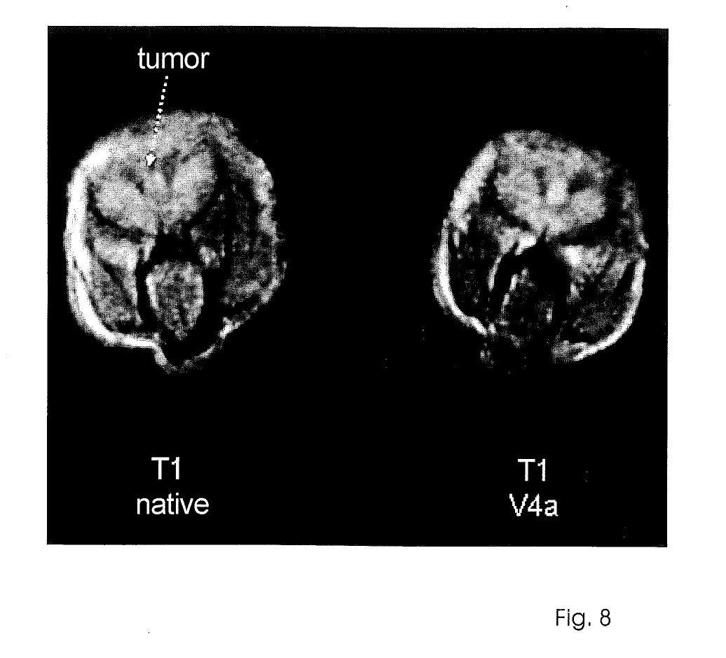

After the intraperitoneal administration of the compound 4A into nude mice with intra-cerebral LN18 brain tumors in the MR tomography a distinct increase of the signal intensity in the brain tumors could be observed; cf. FIG. 8. This increased signal intensity was also seen one day after the administration of the compound in an attenuated form. If the same compound was intraperitoneally administered in the presence of an MMP-2 inhibitor, in the brain tumors only a very minor increase of the signal intensity was observed, which remained only for a short time.

The locations of increased signal intensity in the MRT histologically correspond to the tumor areas. In the CLSM only in these areas stained cell nuclei could be seen. The healthy parenchyma of the brain remained unstained.

1. Compound, comprising:
a first unit (E1), comprising:
a first component providing a permeability through the cell membrane and the nuclear membrane (transportation component),
a metal complexing agent,
a second unit (E2), comprising:
a peptide comprising at least one cleavage side for tumor or virus specific proteases (specificity mediating peptide), and
a third unit (E3), comprising:
at least one metal complexing agent,
wherein E1 is connected to E2 and E2 is connected to E3.

2. Compound according to claim 1, wherein the transportation compound is a peptide (first transportation peptide).

3. Compound according to claim 2, wherein the first transportation peptide comprises a nuclear localization sequence (NLS).

4. Compound according to claim 2, wherein the first transportation peptide comprises a positive net charge.

5. Compound according to claim 2, wherein the first transportation peptide comprises a length of 3 to 20 amino acids.

6. Compound according to claim 2, wherein the first transportation peptide comprises an amino acid sequence selected from the group consisting of: PKKKRKV (SEQ ID No. 1), RRRR (SEQ ID No. 2), and PKKKRKVRRRR (SEQ ID No. 3).

7. Compound according to claim 1, wherein E3 further comprises a second transportation peptide.

8. Compound according to claim 7, wherein the second transportation peptide comprises a nuclear localization sequence (NLS).

9. Compound according to claim 7, wherein the second transportation peptide comprises a positive net charge.

10. Compound according to claim 7, wherein the second transportation peptide comprises a length of 3 to 20 amino acids.

11. Compound according to claim 7, wherein the second transportation peptide comprises an amino acid sequence selected from the group consisting of:

| | |
|---|---|
| PKKKRKV, | (SEQ ID No. 1) |
| RRRR, | (SEQ ID No. 2) |
| and | |
| PKKKRKVRRRR. | (SEQ ID No. 3) |

12. Compound according to claim 1, wherein it comprises a molecular weight of about 2,000 kDa to about 10,000 kDa.

13. Compound according to claim 1, wherein the metal complexing agent comprise tetraazacyclododecane tetraacetic acid (DOTA).

14. Compound according to claim 13, wherein the metal complexing agents have complexed gadolinium (Gd).

15. Compound according to claim 14, wherein the complexed gadolinium (Gd) is radioactively labeled.

16. Compound according to claim 1, wherein E1 and/or E2 and/or E3 further comprises a fluorescent marker.

17. Compound according to claim 1, wherein the specificity mediating peptide comprises a length of 2 to 20 amino acids.

18. Compound according to claim 1, wherein the specificity mediating peptide comprises an amino acid sequence selected from the group consisting of: PLGLA (SEQ ID No. 4) and PLGVA (SEQ ID No. 5).

19. Compound according to claim 16, wherein E1 and/or E3 comprises a spacer which spaces the complexing agent from the fluorescent marker.

20. Compound according to claim 19, wherein the spacer comprises two amino acids.

21. Compound according to claim 20, wherein the spacer comprises two glycine residues.
